SEQUENCE LISTING (1 sequence)

SEQ ID NO: 1 - protein, 416 aa, Saccharomyces pastorianus
  PEPTIDE (1)..(416)
  UNSURE (1)..(416)
MSLSSKLSVQDLDLKDKRVFIRVDFNVPLDGKKITSNQRIVAALPTIKYVLEHHPRYVVLASHLGRPNGERNEKYSLAPVAKELQSLLGKDVTFLNDCVGPEVEAAVKASAPGSVILLENLRYHIEEEGSRKVDGQKVKASKEDVQKFRHELSSLADVYINDAFGTAHRAHSSMVGFDLPQRAAGFLLEKELKYFGKALENPTRPFLAILGGAKVADKIQLIDNLLDKVDSIIIGGGMAFTFKKVLENTEIGDSIFDKAGAEIVPKLMEKAKAKGVEVVLPVDFIIADAFSADANTKTVTDKEGIPAGWQGLDNGPESRKLFAATVAKAKTIVWNGPPGVFEFEKFAAGTKALLDEVVKSSAAGNTVIIGGGDTATVAKKYGVTDKISHVSTGGGASLELLEGKELPGVAFLSEKK

(19) United States
(12) Patent Application Publication
(10) Pub. No.: US 2013/0189298 A1
(43) Pub. Date: Jul. 25, 2013

(54) NOVEL YEAST STRAIN AND THE APPLICATION THEREOF

(71) Applicants: GUO-JANE TSAI, Taipei City (TW); Chien-Hui Wu, New Taipei City (TW)

(72) Inventors: GUO-JANE TSAI, Taipei City (TW); Chien-Hui Wu, New Taipei City (TW)

(73) Assignee: GUO-JANE TSAI, Taipei City (TW)

(21) Appl. No.: 13/418,542

(22) Filed: Mar. 13, 2012

(30) Foreign Application Priority Data
Jan. 20, 2012 (CN) 201210022154.1

(51) Int. Cl. A61K 36/064; C07K 14/395; A61P 3/10; A61P 3/08; A61P 3/04; C12N 1/16; C07H 21/00

(52) U.S. Cl. 424/195.16; 435/255.2; 530/350; 536/23.74

(57) ABSTRACT

The present invention provides an isolated Saccharomyces pastorianus No 54, which is found to be effective in regulating blood glucose levels and fat or its related disease. Therefore, not only could it control the high blood sugar levels in type 1 diabetic patients, but also it would help to control both of the high blood glucose levels and fat of type 2 diabetic patients.

### CROSS REFERENCE TO RELATED APPLICATION

This application claims the priority benefit of China patent application number 201210022154.1, filed on Jan. 20, 2012.

### FIELD OF THE INVENTION

The present invention relates to a novel yeast strain, Saccharomyces pastorianus No. 54, and the application of it and its relative biological materials, and more particularly to the application in regulating blood sugar levels or ameliorating obesity or obesity-related health disorders.

### BACKGROUND OF THE INVENTION

Insulin, produced and released by beta cells in the islets of langerhans, is the key hormone in lowering blood sugar levels. As insulin binds to insulin receptors located in the plasma membrane of muscle cells or fat cells, molecules, such as PI3, PKC, Akt, and p38 MAPK, induced in the downstream signal pathway would be activated and consequently induce the translocation of glucose transporter type 4 (GLUT4) from the vesicles to the cell surface. As a result of a marked increase in cell surface GLUT4, these cells (the muscle cells or fat cells stimulated by insulin) absorb glucose much more rapidly and thus lowering blood sugar level.

Unfortunately, that not only do beta cells in the islets of langerhans fail to produce sufficient insulin but also insulin does not induce downstream signals efficiently on muscle cells or fat cells would cause fasting blood sugar levels exceed 126 mg/dL. It would severely damage tissues and organs that blood sugar levels remains so high all the time. In fact, it would cause retinopathy, nephropathy, neuropathy, and vascular disease, and may even more seriously cause coma or death. Therefore, the pathological features of fasting blood sugar level more than 126 mg/dL is clinically defined as a group of diseases, called diabetes.

There are two main types of diabetes, type 1 diabetes and type 2 diabetes. Type 1 diabetes results from the autoimmune disease allowing the immune response to act against and damage its own islets of langerhans. Therefore, type 1 diabetic patients fail to produce insulin and require to inject insulin for their entire life.

Type 2 diabetic patients could produce insulin normally. However, as a result of insulin resistance, insulin could not efficiently induce downstream signals in type 2 diabetic patients' muscle cells or fat cells and thus fails to lower their blood sugar levels. It is obesity that significantly increases the risk of insulin resistance and type 2 diabetes as obese people have much more adipocytes to secret insulin resistance-related and type 2 diabetes-related cytokines and chemokines. Statistics show that 50% of obese people would get type 2 diabetes; in addition, more than 70% of type 2 diabetic patients are overweight or obese. If these overweight type 2 diabetic patients lose weight, the problem of insulin resistance can be ameliorated to normal or near normal states.

At present, healthy diet is the recommended treatment of type 2 diabetes since it helps to maintain the normal level of blood sugar and maintain healthy body weight; in comparison, if patients are overweight, weight loss is the most effective treatment to restore muscle cells or fat cells to insulin response. However, if neither of these treatments can control the blood sugar, type 2 diabetic patients have to follow their doctors' orders to take oral diabetes drugs or insulin injections everyday.

It is noteworthy that there is, at present, no available drug other than insulin itself to directly activate insulin receptors. In other words, there is no alternative drug stimulating muscle cells or fat cells to absorb glucose much more rapidly and thus lowering blood sugar levels directly. All the oral diabetes drugs can only help to control blood sugar levels more efficiently, including increasing insulin secretion, improving insulin function, inhibiting the decomposition of sugars or delaying the absorption of simple sugars in the gastrointestinal tract.

Therefore, there will be no other drugs able to lower blood sugar when it is as severe as that high doses of insulin combined with oral diabetes drugs could no longer control one's blood sugar (for example, the type 2 diabetic patients with severe insulin resistance); that is, what the patients only could do is to restore insulin resistance by weight loss so that insulin can gradually recover from failing to lower blood sugar levels; however, weight loss is difficult for most people.

Even in the case that high-doses insulin could slightly control blood sugar levels, there are some disadvantages—beta cells in the islets of langerhans may gradually lose its' ability to release insulin while high plasma levels of insulin poorly, for a long period of time, get the blood sugar back to normal (for example, the type 2 diabetic patients with severe insulin resistance). In this situation, weight loss is not enough because patients could no longer secret insulin and hence would lose the ability to lower blood sugar level by themselves for ever.

Therefore, there is a need of providing an alternative substance with effectiveness of lowering blood sugar levels for type 1 and type 2 diabetic patients to replace insulin to lower blood sugar directly. Moreover, there is a need of providing a substance which could induce a series of downstream signals and responses as insulin in order to solve the problem that insulin could not lower blood sugar level in patients with severe insulin resistance. In addition, there is a need of providing a substance with effectiveness of weight loss to reduce insulin resistance so that type 2 diabetic patients with severe insulin resistance could revert to the state to lower blood sugar level effectively and independently.

### SUMMARY OF THE INVENTION

In terms of diabetic patients' need for novel substances with effectiveness of lowering blood sugar levels, the present invention provides a novel yeast strain or its relative biological materials, all of which could lower blood sugar levels and hence could replace insulin to lower blood sugar in type 1 or type 2 diabetic patients.

The present invention also provides a novel yeast strain or its relative biological materials which could induce a series of downstream signals and responses as insulin hence is suitable to lower blood sugar levels in patients with severe insulin resistance.

The present invention further provides a novel yeast strain or its relative biological materials which could lower blood sugar levels in patients with severe insulin resistance and hence avoid the status that such patients would no longer secret insulin resulting from high blood sugar levels for a long period of time.

The present invention still more provides a novel yeast strain or its relative biological materials which could ameliorate obesity or obesity-related health disorders (for example, body weight, body weight gain, adipose tissue weight, hepatic total cholesterol, plasma total cholesterol, plasma HDL-C, plasma LDL-C, or plasma triglyceride etc) and hence help type 2 diabetic patients recover from insulin resistance.

In accordance with an aspect of the present invention, there is provided a novel yeast, Saccharomyces pastorianus No. 54. The yeast is deposited in China Center for Type Culture Collection with the accession number CCTCC M 2011496 on Dec. 31, 2011.

Preferably, the yeast is characterized by regulating blood sugar levels.

Preferably, the yeast regulates said blood sugar levels via its endogenous protein, in which the amino acid sequence is SEQ ID NO: 1; or the yeast regulates blood sugar levels via its endogenous protein, in which the amino acid sequence is SEQ ID NO: 1, and the endogenous protein increases the levels of glucose transporter 4 (GLUT4) or insulin receptor presented on the surface of target cells.

Preferably, the yeast is characterized by ameliorating obesity or obesity-related health disorders.

In accordance with another aspect of the present invention, there is provided a yeast derivative being a derivative or mutant of the yeast according to the present invention, and the yeast derivative is characterized by regulating blood sugar levels.

Preferably, the yeast derivative regulates blood sugar levels via its endogenous protein, in which the amino acid sequence is SEQ ID NO: 1; or

the yeast derivative regulates blood sugar levels via its endogenous protein, in which the amino acid sequence is SEQ ID NO: 1, and the endogenous protein increases the levels of glucose transporter 4 (GLUT4) or insulin receptor presented on the surface of target cells.

Preferably, the derivative is characterized by ameliorating obesity or obesity-related health disorders.

In accordance with third aspect of the present invention, there is provided a yeast extract. The yeast extract is extracted from the yeast according to the present invention or its derivative strain, and the yeast extract is characterized by regulating blood sugar levels or ameliorating obesity or obesity-related health disorders.

In accordance with fourth aspect of the present invention, there is provided a purified yeast extract. The purified yeast extract is purified from the yeast extract according to the present invention, and the purified yeast extract is characterized by regulating said blood sugar levels or ameliorating obesity or obesity-related health disorders.

In accordance with fifth aspect of the present invention, there is provided a purified or synthetic protein comprising amino acid sequence of SEQ ID NO: 1.

Preferably, the purified or synthetic protein is characterized by regulating blood sugar levels; or

the purified or synthetic protein is characterized by regulating blood sugar levels through increasing the levels of glucose transporter 4 (GLUT4) or insulin receptor presented on the plasma membrane of target cells.

In accordance with sixth aspect of the present invention, there is provided a recombinant protein comprising amino acid sequence of similar to SEQ ID NO: 1, wherein one or more than one residue of the recombinant protein is deleted, added or replaced comparing with SEQ ID NO: 1. The recombinant protein is characterized by regulating blood sugar levels.

Preferably, the recombinant protein is characterized by regulating blood sugar levels through increasing the levels of glucose transporter 4 (GLUT4) or insulin receptor presented on the plasma membrane of target cells.

In accordance with seventh aspect of the present invention, there is provided a purified or synthetic nucleic acid encoding a protein comprising amino acid sequence of SEQ ID NO: 1.

Preferably, the protein comprising amino acid sequence of SEQ ID NO: 1 regulates blood sugar levels; or

the protein comprising amino acid sequence of SEQ ID NO: 1 regulates blood sugar levels through increasing the levels of glucose transporter 4 (GLUT4) or insulin receptor presented on the plasma membrane of target cells.

In accordance with eighth aspect of the present invention, there is provided a recombinant nucleic acid comprising nucleotide sequence of similar to the purified or synthetic nucleic acid according to the present invention, wherein one or more than one nucleotide of said recombinant nucleic acid is replaced, deleted, or added comparing with said purified or synthetic nucleic acid, and said recombinant nucleic acid is characterized by encoding a protein regulating blood sugar levels.

Preferably, the protein regulates blood sugar levels through increasing the levels of glucose transporter 4 (GLUT4) or insulin receptor presented on the plasma membrane of target cells.

### BRIEF DESCRIPTION OF THE DRAWINGS

The above objects and advantages of the present invention will become more readily apparent to those ordinarily skilled in the art after reviewing the following detailed description and accompanying drawings, in which:

FIG. 1 is a column chart presenting the effect of each sample, collected from chromatographic columns, on glucose uptake in differentiated 3T3-L1 adipocytes;

FIG. 2 is a protein gel electrophoresis pattern of DW1 sample collected from chromatographic column;

FIG. 3 is a column chart presenting the effect of the 54-kDa protein on signal molecules and proteins in differentiated 3T3-L1 adipocytes; and

FIG. 4 is a result of Western blot analysis presenting the relative amount of glucose transporter 4 in the sample obtained from the plasma membrane of differentiated 3T3-L1 adipocytes stimulated by the 54-kDa protein.

### DETAILED DESCRIPTION OF THE PREFERRED EMBODIMENT

For solving the drawbacks encountered from the prior art, the present invention provides a novel yeast, Saccharomyces pastorianus No. 54 or its yeast derivative not only could which lower blood sugar levels and hence replace insulin to lower blood sugar in type 1 and type 2 diabetic patients effectively, but also which could ameliorate obesity or obesity-related health disorders. The yeast is deposited in China Center for Type Culture Collection (located in Luo-jia-shan, Wuchang, Wuhan, Hubei Province, P.R.C, 430072) with the accession number CCTCC M 2011496 on Dec. 31, 2011.

The following provides detailed instructions for use of the embodiments of the present invention, and the technology and features of the present invention; however, the embodiment is not intended to limit the present invention, and hence a person having ordinarily knowledge in the art may make various changes and modification included within the spirit and scope of the present invention.

Experiment 1: Prepare yeast or its extract with effectiveness of lowering blood sugar levels and ameliorating obesity or obesity-related health disorders.

Inoculate Saccharomyces pastorianus No. 54 into malt extract broth (MEB, Difico Labotories), and incubate it at 25° C. for 48 hours. After activating twice in the same way, inoculate such activated yeast into malt extract broth to a final concentration of 10⁵ CFU/mL and incubate with shaking for 4 days. Centrifuge the yeast culture, remove the supernatant, and then wash the pellet three times with Milli Q water.

Transfer the centrifuged wet yeast cells into an erlenmeyer flask, extract the active substances of such yeast for 2 hours with 0.1 N NH₄OH under the condition of 30° C. and 100 rpm, and then dry the extract through freeze-dried Process to obtain the yeast extract powder.

Experiment 2: Prepare STZ induced type 2 diabetic rats.

Male Sprague-Dawley (SD) rats were housed individually in stainless steel cages, and the temperature in the animal room was maintained at 23±1° C. and the humidity at 40-60%. Such Rats were kept under standard conditions of food and distilled water intake in free-feeding and with a daily photo period of 12 hours light and 12 hours dark. As growing to an average weight of 300 g, these adult rats were treated with nicotinamide (230 mg/kg Body Weight) and streptozotocin (STZ, 65 mg/kg Body Weight in citrate buffer, pH4.6) for a week to induce type 2 diabetes, and then assessed the symptoms of type 2 diabetes by oral glucose tolerance test (OGTT). The rats having the symptoms of diabetes are said STZ-induced type 2 diabetic rats.

Experiment 3: Prepare differentiated 3T3-L1 adipocytes.

Incubate mouse 3T3-L1 preadipocytes (product No. BCRC60159, from Bioresource Collection and Research Center, The Food Industry Research and Development Institute, Hsinchu, Taiwan) for 8 days in High Glucose Dulbecco's Modified Eagle Medium (Gibco) with 10% fetal bovine serum , 10 μg/mL insulin, 1 μM DEX, and 0.5 mM IBMX to obtain differentiated 3T3-L1 adipocytes. The cells were stained with Oil-Red-O dye in solvent(containing 0.3% Oil-Red-O, 60% isopropanol) in the dark at room temperature for 30 minutes to confirm whether the cells have become differentiated 3T3-L1 adipocytes.

Experiment 4: Effect of yeast extract on fasting blood sugar levels or fasting blood insulin levels in type 2 diabetic rats.

The process to test blood sugar levels includes: mixing 104 plasma with the reagent of Glucose Enzymatic Kit (Cat. No. GL 2623, Randox), incubating for 5 minutes at 37° C., reading the absorbance at 500 nm with spectrophotometer, and calculating the concentration of glucose for each sample from glucose standard curve. The process to test blood insulin levels includes: performing the Insulin assay with Rat Insulin ELISA Kit (Mercodia AB, Sweden) and 254 plasma, reading the absorbance at 450 nm with ELISA reader (μ Quant, BIO-TEK, U.S.A), and calculating the concentration of insulin for each sample from insulin standard curve. Table 1 shows the fasting blood sugar levels and fasting blood insulin levels in STZ induced type 2 diabetic rats fed yeast extract for 6 weeks. The average fasting blood sugar levels in diabetic control group is 220.65±20.88 mg/dL, significantly higher than that in normal control group, 192.73±18.56 mg/dL average fasting blood sugar levels (p<0.05). The average fasting blood sugar levels in yeast extract group, type 2 diabetic rats fed yeast extract for 6 weeks, is 194.47±21.02 mg/dL, not significantly different from that in normal control group. It suggests that yeast extract helps diabetic rats lower their blood sugar to normal level.

In comparison, the average fasting blood insulin levels in diabetic control group is 2.10±0.72 mg/L, significantly higher than that in normal control group, 1.44±0.65 mg/L average fasting blood insulin levels (p<0.05). The average fasting blood insulin levels in yeast extract group is 1.28±0.55 mg/L, significantly reduced by 39% (p<0.05) while not significantly different from that in normal control group. It suggests that yeast extract could not only help diabetic rats lower their blood sugar but also help lower blood insulin to normal levels.

TABLE 1

| | Fasting blood sugar | Fasting blood insulin |
|---|---|---|
| | levels (mg/dL) | levels (mg/L) |
| Normal control | 192.73 ± 18.56 | 1.44 ± 0.65 |
| Diabetic control | 220.65 ± 20.88* | 2.10 ± 0.72* |
| Yeast extract | 194.47 ± 21.02** | 1.28 ± 0.55** |

Values were calculated as mean ± SD for rats in each group (n = 7-9).
*p < 0.05 compared with normal control.
**p < 0.05 compared with diabetic control.

Experiment 5: Purify the active substances from the yeast extract.

In order to purify the active substances with effectiveness of lowering blood sugar levels from the yeast extract, yeast extract is separated by DEAE cellulose column or DOWEX 50WX8-200 column and then samples are collected in 7 collection tubes individually, named DC1, DC2, DC3, DW1, DW2, DW3, or DW4 sample.

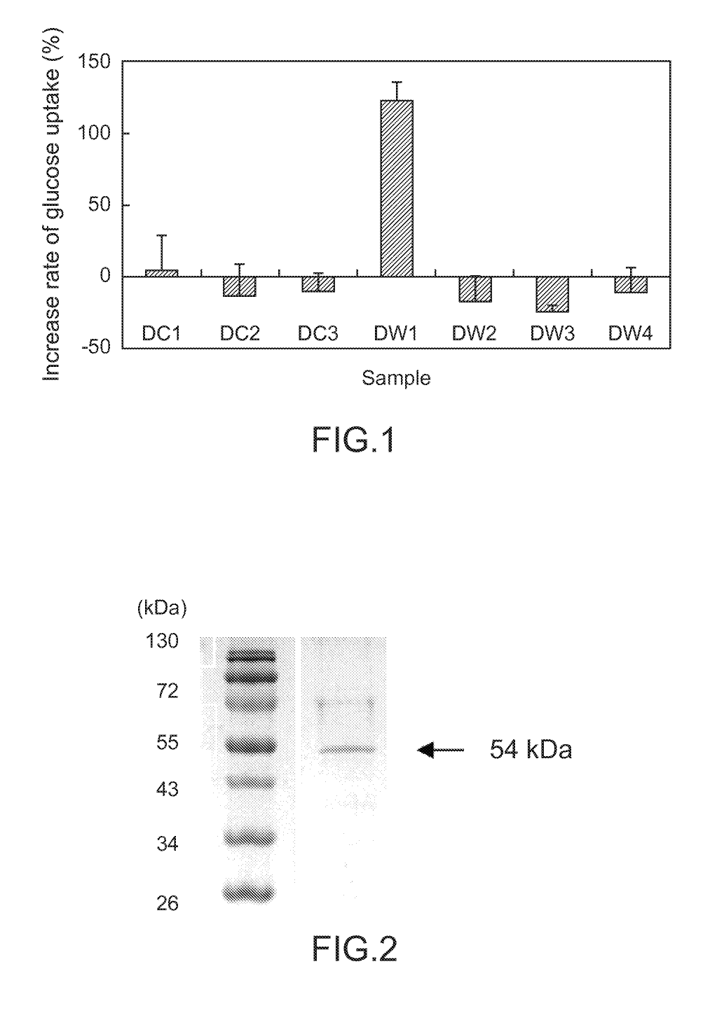

Please see FIG. 1, which is a column chart presenting the effect of each sample, collected from chromatographic columns, on glucose uptake in differentiated 3T3-L1 adipocytes. FIG. 1 shows DC1, DC2, DC3, DW1, DW2, DW3, and DW4 sample on the horizontal axis from left to right in order, and shows increase rate of glucose uptake in differentiated 3T3-L1 adipocytes on the vertical axis. FIG. 1 indicates that no other sample but DW1 sample could increase glucose uptake in differentiated 3T3-L1 adipocytes, and DW1 sample increases glucose uptake by 130%.

Please see FIG. 2, which is a protein gel electrophoresis pattern of DW1 sample. FIG. 2 shows protein marker (mixtures of standard proteins with known molecular weight) on lane 1 and DW1 sample on lane 2, and shows molecular weight on the vertical axis. FIG. 2 indicates that DW1 sample consist mainly of a 54-kDa protein, in which the amino acid sequence is SEQ ID NO: 1.

The sequence of SEQ ID NO: 1 presented as followed

```
    1 MSLSSKLSVQ DLDLKDKRVF IRVDFNVPLD GKKITSNQRI VAALPTIKYV
  51 LEHHPRYVVL ASHLGRPNGE RNEKYSLAPV AKELQSLLGK DVTFLNDCVG
101 PEVEAAVKAS APGSVILLEN LRYHIEEEGS RKVDGQKVKA SKEDVQKFRH
151 ELSSLADVYI NDAFGTAHRA HSSMVGFDLP QRAAGFLLEK ELKYFGKALE
201 NPTRPFLAIL GGAKVADKIQ LIDNLLDKVD SIIIGGGMAF TFKKVLENTE
251 IGDSIFDKAG AEIVPKLMEK AKAKGVEVVL PVDFIIADAF SADANTKTVT
301 DKEGIPAGWQ GLDNGPESRK LFAATVAKAK TIVWNGPPGV FEFEKFAAGT
351 KALLDEVVKS SAAGNTVIIG GGDTATVAKK YGVTDKISHV STGGGASLEL
401 LEGKELPGVA FLSEKK
```

Experiment 6: Effect of the 54-kDa protein in differentiated 3T3-L1 adipocytes.

The process to test the amount of signal molecules or proteins includes washing cells with staining buffer (2% FBS and 0.1% sodium azide in PBS) for 3 times, and then centrifuging at 300×g for 5 minutes. Resuspend cells with 1 mL primary antibody and incubate for 30 minutes at room temperature. After washing cells with staining buffer for 3 times, resuspend cells with 1 mL FITC-conjugated secondary antibody and incubate for 30 minutes at room temperature. Finally, wash cells with staining buffer for 3 times and then detect and analyze the expression of signal molecules or proteins in cells by BD FACSCanto™ Flow Cytometer.

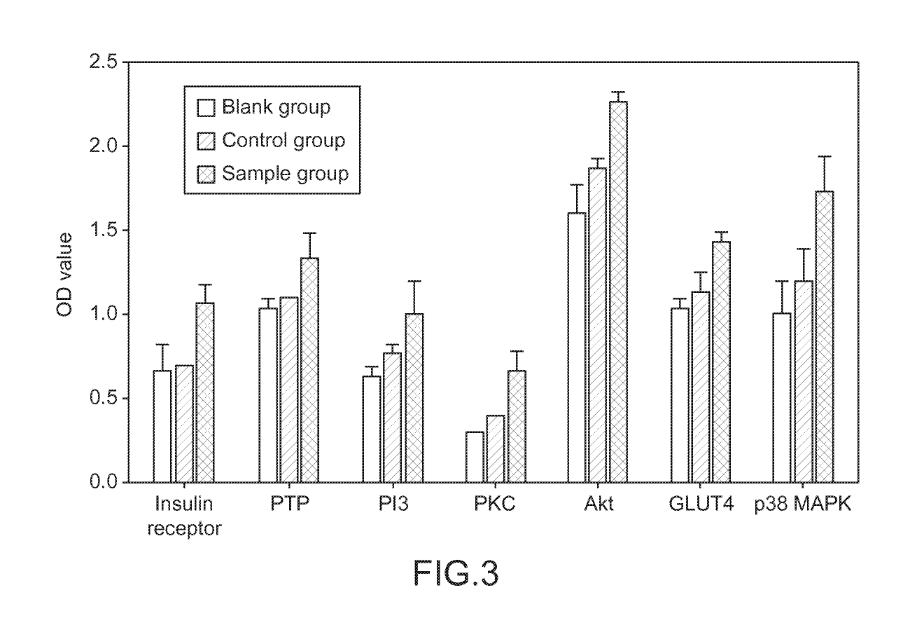

Please see FIG. 3, which is a column chart presenting the effect of the 54-kDa protein on signal molecules and proteins in differentiated 3T3-L1 adipocytes. FIG. 3 shows 7 signal molecules on the horizontal axis from left to right in order, which are activated insulin receptors, activated protein-tyrosine phosphatase (PTP), activated phosphatidylinositol 3-kinase (PI3), activated protein kinase C (PKC), activated protein kinase B (also known as Akt), intracellular glucose transporter 4 (GLUT4), and activated p38 mitogen-activated protein kinase (p38 MAPK). Moreover, there are three groups for all of these signal molecules, which are blank group (no cells; white-filled pattern), control group (normal differentiated 3T3-L1 adipocytes; forward slash-filled pattern), and sample group (differentiated 3T3-L1 adipocytes treated with 54-kDa protein; cross slashes-filled pattern). Furthermore, FIG. 3 shows OD value on the vertical axis. According to the test of the same signal molecule, the higher the OD value is, the more the signal molecules are.

FIG. 3 indicates that all of the activated insulin receptor on plasma membrane, activated PTP, activated PI3, activated PKC, activated Akt, intracellular GLUT4, and activated p38 MAPK are significantly increased if differentiated 3T3-L1 adipocytes are treated with the 54-kDa protein. These results suggest that the 54-kDa protein could induce three insulin-like downstream signal pathways: 1. activating insulin receptor —PTP—PI3—PKC pathway to induce the translocation of vesicles containing GLUT4 to the cell surface; 2. activating insulin receptor —PTP—PI3—Akt pathway to induce the translocation of vesicles containing GLUT4 to the cell surface; 3. activating insulin receptor —p38 MAPK pathway to promote the storage of glucose taken by cells.

The process to test the amount of a plasma membrane protein includes: washing cells twice with Phosphate Buffered Saline (PBS; 137 mM NaCl, 2.7 mM KC, 4.3 mM Na2HPO4, 1.5 mM KH2PO4, pH 7.3), breaking cells with lysis buffer (Tris-HCl, pH 7.4, 1 mM EGTA, 1 mM NaF, 150 mM NaCl, 1 mM PMSF, 5 μg/ml leupeptin, 20 μg/ml aprotinin, 1 mM Na3VO4, 1% Triton X-100) and then centrifuging to obtain the mixture of plasma membrane proteins. Moreover, load the mixture of plasma membrane proteins to the gel, and run the gel at 120V for 60 minutes. Transfer protein bands from gel to PVDF membrane at 100V for 60 minutes. Incubate PVDF membrane with anti-GLUT4 primary antibody and then with HRP-conjugated secondary antibody. Finally, detect GLUT4 protein on PVDF membrane with Enhanced chemiluminescence system (ECL) and then expose to X-ray film.

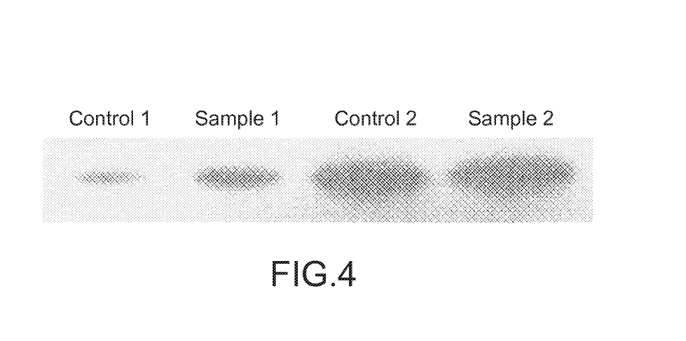

Please see FIG. 4, which is a result of Western blot analysis presenting the relative amount of glucose transporter 4 in the sample obtained from the plasma membrane of differentiated 3T3-L1 adipocytes stimulated by the 54-kDa protein. FIG. 4 shows GLUT4 in 4 groups from left to right in order, which are control 1 group (normal differentiated 3T3-L1 adipocytes), sample 1 group (differentiated 3T3-L1 adipocytes treated with 54-kDa protein), control 2 group (differentiated 3T3-L1 adipocytes treated with insulin), and sample 2 group (differentiated 3T3-L1 adipocytes co-treated with insulin and 54-kDa protein); the darker/thicker the band is, the more the GLUT4 is.

FIG. 4 indicates that the 54-kDa protein itself, without insulin, could increase the amount of GLUT4 in plasma membrane of differentiated 3T3-L1 adipocytes (please see the result of control 1 group and sample 1 group); GLUT4 in differentiated 3T3-L1 adipocytes co-treated with the 54-kDa protein and insulin is more than only treated with insulin (please see the result of control 2 group and sample 2 group). These results suggest that it is the 54-kDa protein in the yeast extract that lowers blood sugar levels and the regulating mechanism of such 54-kDa protein is similar to the mechanism of insulin.

It is noteworthy that not only does the 54-kDa protein extracted from Saccharomyces pastorianus No. 54 have similar function and mechanism with insulin, but also it could induce these reactions independently; therefore, the 54-kDa protein may be able to replace insulin as a novel therapeutic substance. Moreover, according to the result of experiment 3, the 54-kDa protein extracted from Saccharomyces pastorianus No. 54 could lower blood sugar level in type 2 diabetic rats; that is, the 54-kDa protein is suitable for insulin resistant patients as a therapeutic substance to lower blood sugar levels, and hence avoids the situation that such insulin resistant patients may no longer secret insulin because of their long-term high blood sugar levels.

Experiment 7: Effect of yeast extract on body weight in type 2 diabetic rats.

Table 2 shows the initial body weight, the final body weight, and the body weight gain in STZ induced type 2 diabetic rats fed yeast extract for 6 weeks. The average initial body weight in normal control is 321.00±10.68 g, that in diabetic control group is 313.50±8.04 g, and that in yeast extract group is 307.33±8.51 g, and it is not significantly different between groups. Six weeks later, the average final body weight in diabetic control group is 463.17±18.00 g (gaining 149.67±14.81 g of body weight), and is significantly higher than that in normal control group, 445.00±24.54 g average final body weight (gaining 124.00±18.07 g of body weight) (p<0.05). These results indicate that the body weight gain in diabetic rats is significantly higher than that in normal rats. In contrast, the average final body weight in diabetic rats fed yeast extract for 6 weeks, the yeast extract group, is 432.11±27.15 g, significantly lower than that in diabetic control group (p<0.05); the body weight gain in yeast extract group is 124.78 g, and is significantly lower than diabetic control group gaining 149.67 g of body weight by about 16.63%.

TABLE 2

| | Initial body | final body weight | body weight gain |
|---|---|---|---|
| | weight (g) | (g) | (g) |
| Normal control | 321.00 ± 10.68 | 445.00 ± 24.54 | 124.00 ± 18.07 |
| Diabetic control | 313.50 ± 8.04 | 463.17 ± 18.00* | 149.67 ± 14.81* |
| Yeast extract | 307.33 ± 8.51 | 432.11 ± 27.15** | 124.78 ± 24.38** |

Values were calculated as mean ± SD for rats in each group (n = 7-9).
*p < 0.05 compared with normal control.
**p < 0.05 compared with diabetic control.

Experiment 8: Effect of yeast extract on adipose tissue weight in type 2 diabetic rats.

Table 3 shows the adipose tissue weight and the relative adipose tissue weight in STZ induced type 2 diabetic rats fed yeast extract for 6 weeks. “Adipose tissue weight” equal to the sum of “perirenal adipose weight” plus “epididymal adipose weight”. The average adipose tissue weight in normal control group is 12.70±7.38 g, and the average relative adipose tissue weight in normal control group is 2.83±1.57 g/100 g body weight. On the other hand, the average adipose tissue weight in diabetic control group is13.17±1.62 g, and the average relative adipose tissue weight in diabetic control group is 3.02±0.40 g/100 g body weight, and each of them is not significantly different from normal control group. In contrast, the average adipose tissue weight in diabetic rats fed yeast extract for 6 weeks, the yeast extract group, is 10.32±2.08 g, and the average relative adipose tissue weight in such yeast extract group is 2.40±0.47 g/100 g body weight, significantly different from that in diabetic control group (p<0.05). These results on adipose tissue weight are similar to that on body weight, which suggests that the body weight loss affected by yeast extract may be relative with such adipose tissue weight loss.

TABLE 3

| | Adipose tissue weight | Relative adipose tissue weight |
|---|---|---|
| | (g)¹ | (g/100 g body weight) |
| Normal control | 12.70 ± 7.38 | 2.83 ± 1.57 |
| Diabetic control | 13.17 ± 1.62 | 3.02 ± 0.40 |
| Yeast extract | 10.32 ± 2.08*^{,}** | 2.40 ± 0.47*^{,}** |

Values were calculated as mean ± SD for rats in each group (n = 7-9).
¹Adipose tissue weight (g) = perirenal adipose weight (g) + epididymal adipose weight (g)
*p < 0.05 compared with normal control.
**p < 0.05 compared with diabetic control.

Experiment 9: Effect of yeast extract on hepatic total cholesterol in type 2 diabetic rats.

The process to test the hepatic total cholesterol includes: homogenizing tissue with homogeneous machine, centrifuging for 10 minutes at 3000×g and then concentrating with vacuum concentrator to remove organic solvent. Moreover, add the mixture of Chloroform/methanol (2:1 v/v) to a total volume of 10 mL. According to the method of Carlson and Goldford in 1979, mix 10 μL of such hepatic extract solution and 10 μL of Triton X-100, and then concentrate with vacuum concentrator for 1 hours; finally, detect the hepatic total cholesterol with Kits (Cat. No. CH 201, and Cat. No. TR 213, Randox)

Table 4 shows the hepatic total cholesterol in STZ induced type 2 diabetic rats fed yeast extract for 6 weeks. Six weeks later, the average hepatic total cholesterol in normal control group is 45.91±1.46 mg/dL, and the average relative hepatic total cholesterol in normal control group is 9.18±0.29 mg/g liver. On the other hand, the average hepatic total cholesterol in diabetic control group is 45.56±2.69 mg/dL, and the average relative hepatic total cholesterol in diabetic control group is 9.11±0.54 mg/g liver, and each of them is not significantly different from normal control group. In contrast, the average hepatic total cholesterol in diabetic rats fed yeast extract for 6 weeks, the yeast extract group, is 42.56±1.03 mg/dL, and the average relative hepatic total cholesterol in such yeast extract group is 8.51±0.21 mg/g liver, and each of them is significantly different from that in normal control group or in diabetic control group (p<0.05).

TABLE 4

Hepatic total cholesterol

| | (mg/dL) | (mg/g liver) |
|---|---|---|
| Normal control | 45.91 ± 1.46 | 9.18 ± 0.29 |
| Diabetic control | 45.56 ± 2.69 | 9.11 ± 0.54 |
| Yeast extract | 42.56 ± 1.03*^{,}** | 8.51 ± 0.21*^{,}** |

Values were calculated as mean ± SD for rats in each group (n = 7-9).
*p < 0.05 compared with normal control.
**p < 0.05 compared with diabetic control.

Experiment 10: Effect of yeast extract on plasma lipids concentration in type 2 diabetic rats.

The manner to test the total cholesterol: Mix 10 μL plasma with the reagent of Cholesterol Enzymatic Endpoint Method Kit (Cat. No. CH 7945, Randox), incubate for 5 minutes at 37° C., and then read the absorbance at 500 nm with spectrophotometer; calculate the concentration of cholesterol for each sample from cholesterol standard curve. Moreover, the manner to test the high-density lipoprotein cholesterol (HDL-C) and low-density lipoprotein cholesterol (LDL-C): Mix 500 μL plasma with the reagent of Kit (CH 203, Randox), incubate for 10 minutes at room temperature. Centrifuge for 2 minutes at 12000×g immediately, and then separate the supernatant containing HDL-C and the pellet containing LDL-C. Detect the HDL-C and the LDL-C with Cholesterol Enzymatic Endpoint Method Kit (Cat. No. CH201, Randox) rspectively. Furthermore, the manner to test the triglyceride (TG): Mix 10 μL plasma with the reagent of Triglycerides Assay Kit (BXC0272C, Fortress), incubate for 5 minutes at 37° C., and then read the absorbance at 500 nm with spectrophotometer; calculate the concentration of triglyceride for each sample from triglyceride standard curve.

Table 5 shows the plasma lipids concentration in STZ induced type 2 diabetic rats fed yeast extract for 6 weeks. The total cholesterol, HDL-C, LDL-C, and triglyceride in plasma of normal control group are 70.00±7.76 mg/dL, 37.54±8.40 mg/dL, 32.46±9.70 mg/dL, and 127.10±28.50 mg/dL respectively. On the other hand, the total cholesterol, HDL-C, LDL-C, and triglyceride in plasma of diabetic control group are 72.09±4.13 mg/dL, 32.56±4.53 mg/dL, 39.53±3.39 mg/dL, and 143.59±42.04 mg/dL respectively, and each of them is not significantly different from normal control group. In contrast, the total cholesterol, HDL-C, LDL-C, and triglyceride in plasma of diabetic rats fed yeast extract for 6 weeks, the yeast extract group, are74.77±6.29 mg/dL, 38.58±5.45 mg/dL, 36.20±5.89 mg/dL, and 106.72±14.05 mg/dL respectively; among them, each of total cholesterol or LDL-C is not significantly different from diabetic control group, HDL-C is significantly higher than that in diabetic control group (p<0.05), and triglyceride is significantly lower than that in diabetic control group by 25% (p<0.05).

TABLE 5

| | Total | | | |
|---|---|---|---|---|
| | cholesterol | HDL-C | LDL-C | Triglyceride |
| | (mg/dL) | (mg/dL) | (mg/dL) | (mg/dL) |
| Normal | 70.00 ± 7.76 | 37.54 ± 8.40 | 32.46 ± 9.70 | 127.10 ± |
| control | | | | 28.50 |
| Diabetic | 72.09 ± 4.13 | 32.56 ± 4.53 | 39.53 ± 3.39 | 143.59 ± |
| control | | | | 42.04 |
| Yeast extract | 74.77 ± 6.29 | 38.58 ± 5.45** | 36.20 ± 5.89 | 106.72 ± |
| | | | | 14.05** |

Values were calculated as mean ± SD for rats in each group (n = 7-9).
**p < 0.05 compared with diabetic control.

As depicted in Table 1, Table 2, Table 3, Table 4, and Table 5, the novel yeast strain or its relative biological materials of the present invention is effective in type 2 diabetes. However, it is noted that the novel yeast strain or its relative biological materials of the present invention is also effective in type 1 diabetes.

Based on above results, the 54-kDa protein extracted from Saccharomyces pastorianus No. 54 has similar function and mechanism with insulin and could induce these reactions independently; therefore, the 54-kDa protein may be able to replace insulin as a novel therapeutic substance. Moreover, such said 54-kDa protein could lower blood sugar level in type 2 diabetic rats; that is, it is suitable for insulin resistant patients as a therapeutic substance to lower blood sugar levels, and hence avoid the situation that such insulin resistant patients may no longer secret insulin because of their long-term high blood sugar levels. In addition, Saccharomyces pastorianus No. 54 or its relative biological materials could ameliorate obesity or obesity-related health disorders, including weight loss, reducing adipose tissue, lowering the concentrations of hepatic total cholesterol, increasing the concentration of plasma HDL-C, and lowering the concentration of plasma triglyceride etc; therefore, such Saccharomyces pastorianus No. 54 or its relative biological materials is probability able to reduce insulin resistance in type 2 diabetic patients.

In other words, instead of insulin, the present invention provides a novel therapeutic substance which is suitable for patients with severe insulin resistant to lower blood sugar levels and hence avoids the situation that such insulin resistant patients may no longer secret insulin because of their long-term high blood sugar levels; more importantly, such novel therapeutic substance makes obese people lose weight and reduce adipose tissue, and therefore, makes obese type 2 diabetic patients be able to lower and regulate blood sugar by themselves again as insulin resistance has been reduced.

That is, the present invention provides Saccharomyces pastorianus No. 54 or its relative biological materials (for example: 1. yeast derivative which is obtained from said yeast after treatment of ultraviolet ray, mutagens, or other mutation methods; 2. yeast extract obtained from said yeast or said yeast derivative; 3. the composition containing said yeast extract; 4. purified yeast extract obtained from said yeast extract after purification; 5. protein, wherein the amino acid sequence is SEQ ID NO: 1; 6. recombinant protein, wherein one or more than one residue of said recombinant protein is deleted, added or replaced comparing with SEQ ID NO: 1; 7. the composition containing said protein or said recombinant protein; 8. nucleic acid encoding a protein in which the amino acid sequence is SEQ ID NO: 1; or 9. recombinant nucleic acid, wherein one or more than one nucleotide is replaced, deleted, or added comparing with said nucleic acid.) could effectively regulate and control blood sugar levels in various types of diabetic rats, and may improve the development of basic and clinical research in diabetes in medical and pharmaceutical fields.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A novel yeast, Saccharomyces pastorianus No. 54, in which said novel yeast is deposited in China Center for Type Culture Collection with the accession number CCTCC M 2011496.

2. The yeast according to claim 1, wherein said yeast is characterized by regulating blood sugar levels.

3. The yeast according to claim 2, wherein said yeast regulates said blood sugar levels via its endogenous protein, in which the amino acid sequence is SEQ ID NO: 1; or
wherein said yeast regulates said blood sugar levels via its endogenous protein, in which the amino acid sequence is SEQ ID NO: 1, and said endogenous protein increases the levels of glucose transporter 4 (GLUT4) or insulin receptor presented on surface of target cells.

4. The yeast according to claim 1, characterized by ameliorating obesity or obesity-related health disorders.

5. A yeast derivative being a derivative or mutant of said yeast according to claim 1 wherein said yeast derivative is characterized by regulating blood sugar levels.

6. The yeast derivative according to claim 5, wherein said yeast derivative regulates blood sugar levels via its endogenous protein, in which the amino acid sequence is SEQ ID NO: 1; or
wherein said yeast derivative regulates blood sugar levels via its endogenous protein, in which the amino acid sequence is SEQ ID NO: 1, and said endogenous protein increases the levels of glucose transporter 4 (GLUT4) or insulin receptor presented on the surface of target cells.

7. The yeast derivative according to claim 5, characterized by ameliorating obesity or obesity-related health disorders.

8. A yeast extract, said yeast extract is extracted from said yeast according to claim 1 or its derivative strain, wherein said yeast extract is characterized by regulating blood sugar levels or ameliorating obesity or obesity-related health disorders.

9. A purified yeast extract, said purified yeast extract is purified from said yeast extract according to claim 8, wherein said purified yeast extract is characterized by regulating said blood sugar levels or ameliorating obesity or obesity-related health disorders.

10. A purified or synthetic protein comprising amino acid sequence of SEQ ID NO: 1.

11. The purified or synthetic protein according to claim 10, wherein said purified or synthetic protein is characterized by regulating blood sugar levels; or
wherein said purified or synthetic protein is characterized by regulating blood sugar levels through increasing the levels of glucose transporter 4 (GLUT4) or insulin receptor presented on the plasma membrane of target cells.

12. A recombinant protein comprising amino acid sequence of similar to SEQ ID NO: 1, wherein one or more than one residue of said recombinant protein is deleted, added or replaced comparing with SEQ ID NO: 1, and said recombinant protein is characterized by regulating blood sugar levels.

13. The recombinant protein according to claim 12, wherein said recombinant protein is characterized by regulating blood sugar levels through increasing the levels of glucose transporter 4 (GLUT4) or insulin receptor presented on the plasma membrane of target cells.

14. A purified or synthetic nucleic acid encoding a protein comprising amino acid sequence of SEQ ID NO: 1.

15. The purified or synthetic nucleic acid according to claim 14, wherein said protein comprising amino acid sequence of SEQ ID NO: 1 regulates blood sugar levels; or
wherein said protein comprising amino acid sequence of SEQ ID NO: 1 regulates blood sugar levels through increasing the levels of glucose transporter 4 (GLUT4) or insulin receptor presented on the plasma membrane of target cells.

16. A recombinant nucleic acid comprising nucleotide sequence of similar to the purified or synthetic nucleic acid according to claim 14, wherein one or more than one nucleotide of said recombinant nucleic acid is replaced, deleted, or added comparing with said purified or synthetic nucleic acid, and said recombinant nucleic acid is characterized by encoding a protein regulating blood sugar levels.

17. The recombinant nucleic acid according to claim 16, wherein said protein regulates blood sugar levels through increasing the levels of glucose transporter 4 (GLUT4) or insulin receptor presented on the plasma membrane of target cells.
